(19) United States
(12) Patent Application Publication
(10) Pub. No.: US 2008/0140124 A1
(43) Pub. Date: Jun. 12, 2008

(54) Spinal rod transverse connector system

(76) Inventors: Dong Myung Jeon, Draper, UT (US); Patrick Dennis Moore, West Jordan, UT (US); Sang K. Lee, Seoul (KR)

Correspondence Address: MORRISS OBRYANT COMPAGNI, P.C., 734 EAST 200 SOUTH, SALT LAKE CITY, UT, 84102, US

(21) Appl. No.: 11/699,873

(22) Filed: Jan. 30, 2007

Related U.S. Application Data
(60) Provisional application No. 60/873,425, filed on Dec. 7, 2006.

(51) Int. Cl. A61B 17/58; A61B 17/56

(52) U.S. Cl. 606/278; 606/246; 606/264; 606/300

(57) ABSTRACT

A transverse connector system for coupling elongate elements to each other in a spinal fixation system. A trans-connector plate with at least one slot-like passage may have top and bottom elongated concave surface configurations. A cylindrical component of a coupling member may be configured to couple with an elongate fixation element by way of a helical slot. A male component on the coupling member may be inserted through the slot like passage and a locking member may be secured thereon to position and orient the coupling member. A second coupling member and locking member may be utilized through a passage in the trans-connector plate, which may be a second slot-like passage. Each locking member cooperates with its respective coupling member to prevent uncoupling, and each may be positioned at a desired lateral position along a slot-like passage.

### CROSS-REFERENCE TO RELATED APPLICATION

This application claims the benefit of U.S. Provisional Application No. 60/873,425, filed Dec. 7, 2006, the disclosure of which is incorporated herein by reference in its entirety.

### TECHNICAL FIELD

The present invention relates to a device for spinal fixation, and in particular to a transverse connector system for coupling spinal rods, plates, or other elongate members.

### BACKGROUND

It is often necessary to surgically treat spinal disorders such as scoliosis. Numerous systems for use in spinal correction and fixation have been disclosed. These systems usually include a pair of elongate members, typically either rods or plates, placed along the vertebral column. For the sake of simplicity, the term “rod” is used throughout to refer to any such elongate member. Each rod is attached to the spine with various attachment devices. These attachment devices may include, but are not limited to, pedicle screws, plates, transverse process hooks, sublaminar hooks, pedicle hooks, and other similar devices.

It is also well known that the strength and stability of a dual rod assembly can be increased by coupling two rods with a cross-brace or transverse connector which extends substantially horizontal to the longitudinal axes of the rods, typically across the spine. The simplest situation in which a transverse connector may be used occurs when the two rods are geometrically aligned parallel to each other in all three dimensions. In such an alignment, there is no convergence or divergence between the rods in the medial-lateral direction, the two rods have the same orientation with respect to the coronal plane (viewed in the anterior-posterior direction), the rods are coplanar from a lateral view, and the two rods are located a uniform distance from each other.

However, the two rods are rarely three dimensionally geometrically aligned in clinical situations. There are several ways to address the variations of geometrical alignment. First, one or both of the rods can be bent to accommodate a transverse connector. However, any bending in either of the rods can adversely affect the fixation to the spine and compromise the clinical outcome. Furthermore, such bending can also adversely affect the mechanical properties of the rods. Alternatively, a transverse connector can be bent so that disturbance to the rod positioning is minimized. As is the case with bending of the rods, the mechanical properties of the transverse connector may be compromised by such bending.

In order to address this issue, transverse connectors with some adjustability have been designed to adapt for variations from geometrical alignment. However, most of such connectors are multi-piece systems which can be difficult to assemble and use in the surgical environment. For example, U.S. Pat. No. 5,980,523, the disclosure of which is incorporated herein by reference in its entirety, discloses a multi-piece transverse connector for spinal rods that can accommodate converging or diverging rods. However, accidental disassembly of this type of connector by the surgeon is possible.

Other connectors which are one-piece designs do not allow for adjustments to compensate for all three modes in which there may be variation from geometrical alignment: convergence or divergence, non-coplanar rods, and variability in rod separation distances. For example, U.S. Pat. No. 5,947,966, the disclosure of which is incorporated by reference herein, discloses a device for linking adjacent spinal rods. In one embodiment, the device includes two members that are movable with respect to one another to accommodate different rod separation distances. A pin on one member engages a groove on the other member to provisionally couple the two members, thereby preventing a surgeon from separating the two members. Because the pin is sized to exactly fit the groove, no movement of the pin transverse to the longitudinal axis of the groove is possible. As a result, the devices similar to the '966 patent device cannot accommodate non-coplanar rods or adjust for rod convergence or divergence.

Thus, there exists a need for an improved transverse connector utilized for connecting or coupling elongate fixation elements to each other in a spinal fixation system that allows for adjustment in translational and/or rotational placement to adjust for convergence or divergence, non-coplanarity, and variability in separation between the elongate fixation elements.

### SUMMARY

In one illustrative embodiment, the present invention includes a transverse connector system for coupling first and second elongate spinal fixation elements which may have different three dimensional orientations to each other. A trans-connector plate with at least one slot-like passage may have top and bottom elongated concave surface configurations. A cylindrical component of a coupling member may be configured to couple with an elongate fixation element by way of a helical slot. A male component on the coupling member may be inserted through the slot-like passage, and a locking member may be secured thereon to position and orient the coupling member. A second coupling member and locking member may be utilized through a passage in the trans-connector plate, which may be a second slot-like passage. Each locking member cooperates with its respective coupling member to prevent uncoupling, and each may be positioned at a desired lateral position along a slot-like passage. By using separately adjustable coupling members and locking members, different separation distances and orientations between elongate fixation elements in a spinal fixation system may be accommodated.

Additional embodiments, examples, advantages, and objects of the present invention will be apparent to those of ordinary skill in the art from the following specification.

### DESCRIPTION OF THE DRAWINGS

It will be appreciated by those of ordinary skill in the art that the elements depicted in the various drawings are not to scale, but are for illustrative purposes only. The nature of the present invention, as well as other embodiments of the present invention may be more clearly understood by reference to the following detailed description of the invention, to the appended claims, and to the several drawings attached hereto.

FIG. 1 is a cut-away side view of one illustrative embodiment of a transverse-connector system in accordance with the principles of the present invention.

FIG. 2 is a bottom view of the transverse-connector system of FIG. 1.

FIG. 3 is a top view of the transverse-connector system of FIGS. 1 and 2.

FIG. 4A is a side view of one illustrative embodiment of a coupling member for use in a transverse connector system in accordance with the present invention.

FIG. 4B is a front view of the coupling member of FIG. 4A.

FIG. 5A shows a cut-away view of an illustrative embodiment of a locking member for use in a transverse-connector system in accordance with the present invention.

FIG. 5B is a top view of the locking member of FIG. 5A.

FIG. 5C is a bottom view of the locking member of FIGS. 5A and 5B.

FIG. 6 is a partial cut-away side view of the coupling member of FIGS. 4A and 4B together with the locking member of FIGS. 5A, 5B and 5C in a transverse connector system in accordance with the present invention.

### DETAILED DESCRIPTION

For the purposes of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

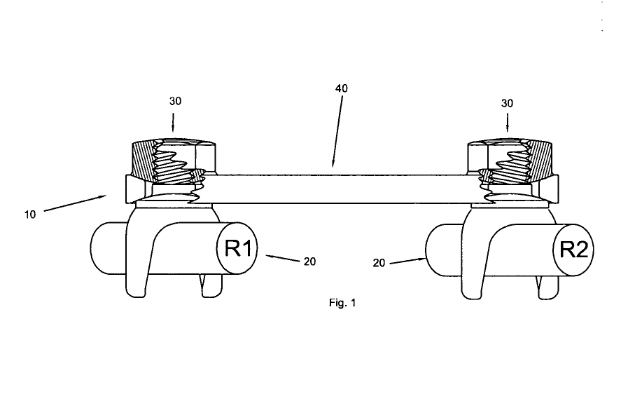

Referring generally to FIG. 1, a transverse connector system 10 in accordance with the present invention is depicted. Transverse-connector 10 system may include one or more coupling members 20, a counterpart internally threaded female locking member 30, and at least one trans-connector plate member 40.

Transverse connector system 10 may be used for coupling a first elongate fixation element R1 to a second elongate fixation element R2. First and second elongate fixation elements R1 and R2 may be cylindrical rods, rectangular bars, plates, or any other device suitable for spinal fusion. In a spinal fixation application, first elongate fixation element R1 may extend along one side of the vertebral column, attached thereto by separate attachment elements. Similarly, second elongate fixation element R2 may extend along the opposite side of the vertebral column, attached thereto by separate attachment elements.

The components of transverse-connector system 10 can be made of any sturdy biocompatible material suitable for an orthopedic application. Suitable materials may include titanium, stainless steel, and alloys containing the same. Where the components of transverse-connector system 10 are constructed from metallic materials, the materials may be similar, or identical to, the metallic materials used for the elongate fixation elements to avoid galvanic (mixed-metal) corrosion.

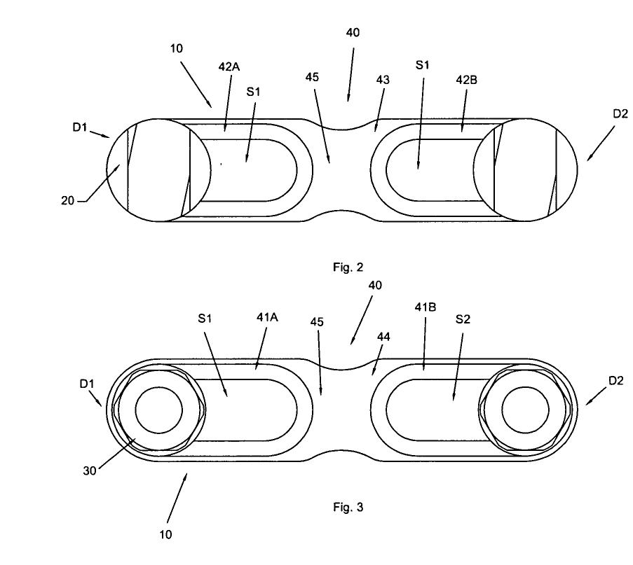

As shown in FIGS. 1, 2 and 3, the trans-connector plate 40 may be formed as a generally planar member with an upper surface 44 and a lower surface 43. It may include a solid section forming a medial bridge 45, from which two wings extend outwards opposite one another to distal ends D1 and D2. Each wing may contain an elongated slot S1 or S2 passing therethrough from the upper surface 44 to the lower surface 43. Surrounding each slot S1 or S2, a concave inset 41A or 41B may be formed in the upper surface. In some embodiments, insets 41A and 41B may have a concave spherical configuration. Similarly, a concave inset 42A or 42B may be formed in the lower surface 43 surrounding each slot S1 or S2. In some embodiments, insets 41A and 41B may have a concave spherical configuration. The surface of the concave insets 41A, 41B, 42A, and 42B may be roughened or knurled to increase engagement to other components of the system 10, as discussed further herein.

The inclusion of medial bridge 45 in trans-connector plate 40 may provide additional strength and stability to the system 10. However, it will be appreciated that in some alternate embodiments, a trans-connector plate 40, which lacks a medial bridge 45 and includes a single slot formed by the union of slots S1 and S2, may be used. It will be further appreciated that although embodiments with two opposite wings and slots S1 and S2 are depicted, that embodiments including multiple slots in a single wing, or including different numbers of wings (such as 3, 4, or more wings containing slots) may be used and are within the scope of the present invention.

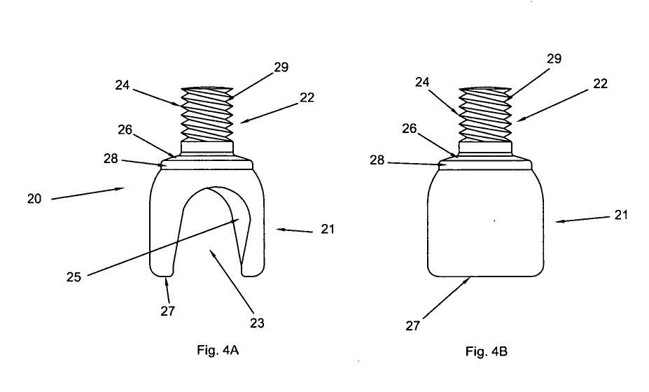

Turning to FIGS. 4A and 4B, a coupling member 20 in accordance with the principles of the present invention is depicted. Coupling member 20 includes a lower portion 21 which may have a generally cylindrical shape. A helical slot 23 extends upwards into the body of the lower portion 21 from a bottom end 27. The walls 25 of the slot 23 are thereby configured to couple with an elongate fixation element, such as a spinal fixation rod, when the coupling member 20 is rotated about a rod inserted into the slot 23 at bottom end 27. Slot 23 may have a helical rotation angle of at least about 11 degrees about the axis of the cylindrical component. The interior surface of walls 25 may be roughened or knurled for increased contact with an inserted elongate member.

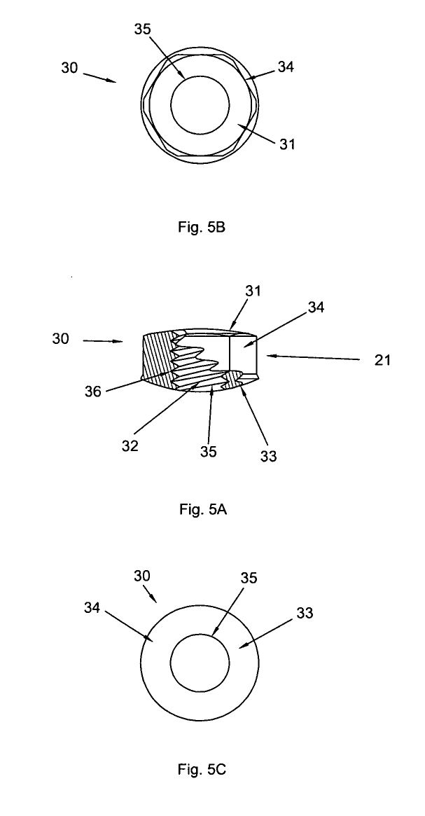

Coupling member 20 also includes a top portion 22 formed at a top end of lower portion 21. Top portion 22 includes a linking element 29, which may be a post with threads 24 for receiving an internally threaded locking member 30 (FIG. 5A). Between upper portion 22 and lower portion 21, the coupling member 20 has a convex surface 26. Convex surface 26 may be formed as a protrusion 28 disposed on the top surface of lower portion 21 and surrounding the linking element 29. Upon installation, linking element 29 passes through a slot S1 or S2 of a trans-connector plate 40 and convex surface 26 contacts the concave inset 42A or 42B formed in the bottom surface of the trans-connector plate 40. Convex surface 26 may be roughened or knurled to increase the security of the contact with a trans-connector plate 40.

FIGS. 5A, 5B and 5C depict an internally threaded locking member 30 for use in a transverse connector system 10, in accordance with the present invention. Locking member 30 may be generally formed as an internally threaded nut. A bore 32 extends from an upper surface 31 to a lower convex surface 33. The internal wall 36 of bore 32 may include threads 35 for securing the locking member 30 to the coupling member 20. The external sidewall 34 of locking member 30 may have planar portions to allow for interaction with a turning tool, such as a wrench. It will, of course, be appreciated that alternative configurations, where a tool print is disposed on the upper surface 31 of the locking member 30 may be used.

Upon installation, the locking member 30 may be threadably attached to a linking element 29 of a coupling member 20 that passes through a slot S1 or S2 of a trans-connector plate 40 and lower convex surface 33 brought into contact with a concave inset 41A or 41B formed in the upper surface of the trans-connector plate 40. Lower convex surface 33 may be roughened or knurled to increase the security of the contact with a trans-connector plate 40.

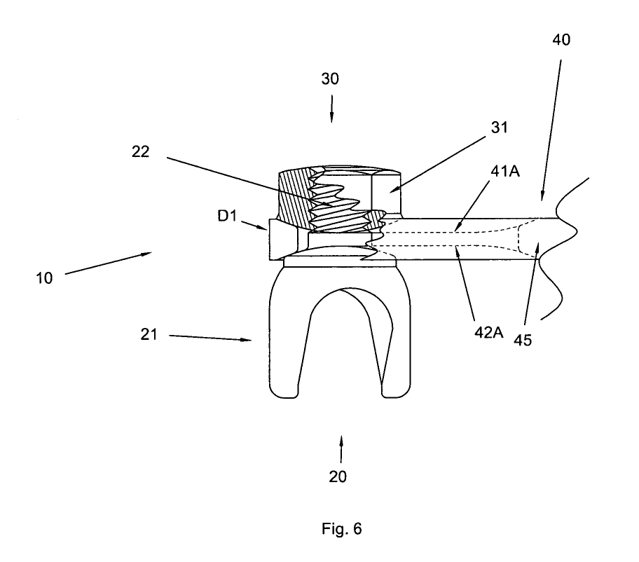

FIG. 6 depicts an enlarged side view of one side of a transverse-connector system 10 in a partial cut-away to highlight the interfaces between the trans-connector plate 40, locking member 30, and coupling member 20. For use in securing elongated members, such as spinal rods, the linking element 29 of a coupling member 20 is inserted into a slot S1 or S2 of a trans-connector plate 40. A locking member 30 is then threaded onto the linking element 29. The convex surface 28 of the coupling element 20 is drawn towards the concave inset 42A or 42B of the lower surface 43 of the trans-connector plate and the lower convex surface 33 of the locking member 30 is drawn towards the concave inset 41A or 41B of the upper surface 44 of the trans-connector plate 40.

An elongated member, such as a spinal fixation rod, may be placed in the helical slot 23 of the coupling member 20. The joined coupling member 20 and locking member 30 are positioned in the desired lateral position along the slot S1 or S2. The locking member 30 is tightened on the linking element 29. The coupling member 20 is thereby rotated, drawing the elongated member R into slot 23 to secure therein. The trans-connector plate 40 is secured between the coupling member 20 and locking element 30, as concave insets 41 and 42 are compressed between the lower convex surface 33 and convex surface 26. This process may be repeated for a second elongated member R using the second slot S1 or S2 of the trans-connector plate 40 with a second coupling member 20 and locking element 30.

In this fashion, connector systems 10 in accordance with the present invention include adjustability for attachment to elongate elements R1 and R2 that are not coplanar along their entire axes. Adjustability is provided by the lateral movement of the coupling member 20/locking element 30 construct in the slots S1 and S2. Additionally, since each coupling member 20 is secured to an elongated member R by rotation of a separate individual cylinder, the long axis of each elongated member R may have a different angular relationship to the system 10, while being secured thereto.

While the present invention has been shown and described in terms of preferred embodiments thereof, it will be understood that this invention is not limited to any particular embodiment and that changes and modifications may be made without departing from the true spirit and scope of the invention as defined and desired to be protected.

What is claimed is:

1. A transverse connector system for coupling elongate elements to each other as part of a spinal fixation system, comprising
a trans-connector plate with at least one slot-like passage;
a first coupling member comprising a body having a helical slot with an opening at a bottom end of the first coupling member, and a linking element disposed on a top end, the linking element sized for insertion through the at least one slot-like passage; and
a first locking member configured to secure to the linking element of the first coupling member position, and orient the coupling member.

2. The transverse connector system of claim 1, wherein the trans-connector plate further comprises a second slot-like passage.

3. The transverse connector system of claim 2, wherein the trans-connector plate comprises two opposite lateral wings, with the at least one slot-like passage disposed in a first wing and the second slot-like passage disposed in the second wing.

4. The transverse connector system of claim 3, wherein the trans-connector plate comprises a medial bridge with the at least one slot-like passage and the second slot-like passage symmetrically disposed in the opposite lateral wings.

5. The transverse connector system of claim 1, wherein the trans-connector plate further comprises an elongated concave surface configuration in a top surface thereof, around the at least one slot-like passage.

6. The transverse connector system of claim 5, wherein a surface of the elongated concave surface configuration in the top surface of the trans-connector plate is roughened or knurled.

7. The transverse connector system of claim 1, wherein the trans-connector plate further comprises an elongated concave surface configuration in a bottom surface thereof, around the at least one slot-like passage.

8. The transverse connector system of claim 7, wherein a surface of the elongated concave surface configuration in the bottom surface of the trans-connector plate is roughened or knurled.

9. The transverse connector system of claim 1, wherein the body of the first coupling member is generally cylindrical.

10. The transverse connector system of claim 9, wherein the helical slot of the first coupling member has a helical rotation angle of at least about 11 degrees about an axis of the generally cylindrical body.

11. The transverse connector system of claim 1, wherein the body of the first coupling member further comprises a convex surface at an upper end thereof.

12. The transverse connector system of claim 11, wherein the convex surface at an upper end of the body of the first coupling member is roughened or knurled.

13. The transverse connector system of claim 1, wherein the first locking member comprises an internally threaded fastener.

14. The transverse connector system of claim 13, wherein the internally threaded fastener comprises a planar sidewall for interacting with a turning tool.

15. The transverse connector system of claim 1, wherein the first locking member has a convex bottom surface that is roughened or knurled.

16. The transverse connector system of claim 1, further comprising a second coupling member comprising a body having a helical slot with an opening at a bottom end of the first coupling member, and a linking element disposed on a top end.

17. The transverse connector system of claim 16, wherein the first coupling member and second coupling member may be separately positioned at desired lateral positions with respect to the trans-connector plate, with their respective linking elements each passing through a slot-like passage therein.

18. A transverse connector system for a spinal fixation system, comprising
a trans-connector plate with at least one slot-like passage;
a first coupler comprising a body having a helical slot opening at a bottom end thereof and a linking element disposed at top thereof, the linking element sized for insertion through the at least one slot-like passage; and
a first fastener configured to secure to the linking element of the first coupler thereby securing the first coupler to the trans-connector plate.

19. The transverse connector system of claim 18, further comprising:
a second coupler comprising a body having a helical slot opening at a bottom end thereof and a linking element disposed at top thereof, the linking element sized for insertion through the at least one slot-like passage; and
a first fastener configured to secure to the linking element of the first coupler thereby securing the first coupler to the trans-connector plate.

20. The transverse connector system of claim 19, wherein the trans-connector plate further comprises a second slot-like passage.

21. The transverse connector system of claim 20, wherein the trans-connector plate comprises two opposite lateral wings, with the at least one slot-like passage disposed in a first wing and the second slot-like passage disposed in the second wing.

22. The transverse connector system of claim 20, wherein the first coupler and second coupler may be separately positioned at desired lateral positions in the at least one slot-like passage and the second slot-like passage.

23. The transverse connector system of claim 18, wherein the trans-connector plate further comprises an elongated concave surface configuration in a top surface thereof, around the at least one slot-like passage.

24. The transverse connector system of claim 23, wherein the first fastener has a convex bottom surface that interfaces with the elongated concave surface configuration in the top surface of the trans-connector plate upon installation.

25. The transverse connector system of claim 18, wherein the trans-connector plate further comprises an elongated concave surface configuration in a bottom surface thereof, around the at least one slot-like passage.

26. The transverse connector system of claim 25, wherein the body of the first coupler has a convex surface at an upper end thereof that interfaces with the elongated concave surface configuration in the bottom surface of the trans-connnector plate upon installation.

27. The transverse connector system of claim 18, wherein the helical slot of the first coupler has a helical rotation angle of at least about 11 degrees about an axis of the generally cylindrical body.
